(19) United States
(12) Patent Application Publication
(10) Pub. No.: US 2023/0390570 A1
(43) Pub. Date: Dec. 7, 2023

(54) DEVICES AND METHODS FOR STIMULATING AN AURICULAR BRANCH OF A VAGUS NERVE

(71) Applicant: The Alfred E. Mann Foundation for Scientific Research, Valencia, CA (US)

(72) Inventors: Brian Michael Shelton, Altadena, CA (US); Neil Talbot, La Crescenta, CA (US); Brian Dearden, Pasadena, CA (US)

(21) Appl. No.: 18/170,199

(22) Filed: Feb. 16, 2023

Related U.S. Application Data
(60) Provisional application No. 63/349,974, filed on Jun. 7, 2022.

(51) Int. Cl. A61N 2/00; H01F 7/06; A61N 2/02

(52) U.S. Cl. CPC A61N 2/006; H01F 7/064; A61N 2/02

(57) ABSTRACT

A stimulation device includes a first transmitting coil and a drive circuit electrically coupled to the first transmitting coil and configured to drive the first transmitting coil. The first transmitting coil is configured, when the device is in proximity to a person's ear and the first transmitting coil is driven with a time-varying electric current providable by the drive circuit, to generate at least part of a magnetic field providing a time-varying magnetic flux across an auricular branch of a Vagus Nerve (ABVN) sufficient to activate the ABVN.

### CROSS REFERENCE TO RELATED APPLICATION

This application claims priority to and the benefit of U.S. Provisional Application No. 63/349,974, filed Jun. 7, 2022, the entire content of which is hereby incorporated by reference.

### BACKGROUND

### 1. Field

The present disclosure relates to devices and methods for stimulating the Vagus Nerve (VN), and more particularly, to devices and methods for stimulating the Auricular Branch of the Vagus Nerve.

### 2. Description of the Related Art

The Vagus Nerve (VN) provides a pathway for signals between the brain and other organs of the human body, including the heart and digestive tract, and plays a role in transmitting afferent and efferent signals regarding various involuntary functions of the body, such as digestion, heart rate and blood pressure, immune system, mood, and skin and muscle sensations. Activating the VN by providing electrical stimulation to the VN may improve the performance of some of these functions and may be utilized to treat certain medical issues, such as depression, epilepsy, and inflammatory bowel disease. This stimulation may be provided via the use of an implant with its attendant surgical and biocompatibility risks. There is, therefore, a need to improve technology relating to stimulating the VN.

The description of the related art in this Background section is included to provide a background understanding of the present disclosure, and thus, is not an admission that what is described is prior art.

### SUMMARY

In an aspect, the technology relates to a stimulation device. In another aspect, the technology relates to a method of stimulating an auricular branch of a Vagus Nerve (ABVN).

Additional aspects will be set forth in part in the description which follows and, in part, will be apparent from the description, or may be learned by practice of the presented embodiments of the disclosure.

In an example, the device includes: a first transmitting coil; and a drive circuit electrically coupled to the first transmitting coil and configured to drive the first transmitting coil, the first transmitting coil being configured, when the device is in proximity to a person's ear and the first transmitting coil is driven with a time-varying electric current providable by the drive circuit, to generate at least part of a magnetic field providing a time-varying magnetic flux across the ABVN sufficient to activate the ABVN.

In an example, the device includes a second transmitting coil, the first and second transmitting coils being positionable on different sides of a part of the person's ear and configured, when positioned on the different sides of the part of the person's ear and driven with respective time-varying electric currents, to generate at least part of the magnetic field. In another example, an axis (e.g., a virtual axis) of the first transmitting coil is aligned or align-able with an axis (e.g., a virtual axis) of the second transmitting coil. In another example, an axis (e.g., a virtual axis) of the first transmitting coil and an axis (e.g., a virtual axis) of the second transmitting coil are set or adjustable to cross (or intersect) and form an angle between 90 degrees and 180 degrees. In another example, the device is configured to directionally control at least part of the magnetic field by controlling one or both of the relative positions and the relative orientations of the first and second transmitting coils with respect to each other. In another example, the device is configured to generate and directionally control at least part of the magnetic field by: driving the first transmitting coil and selectively driving the second transmitting coil; or driving the first transmitting coil and differentially driving the second transmitting coil. In another example, the device further includes a ferrite piece, the ferrite piece being positionable on a different side of a part of the person's ear to the first transmitting coil such that a magnitude of the magnetic field across the ABVN is greater than when the ferrite piece is excluded. In another example, the ferrite piece is shaped to at least partially extend around the part of the person's ear. In another example, the first transmitting coil is shaped such that two ends of the first transmitting coil are positionable on different sides of a part of the person's ear. In another example, the first transmitting coil has an open-ringed or horseshoe shape. In another example, the device is configured such that a distance between the two ends of the first transmitting coil is adjustable. In another example, the device further includes a second transmitting coil and a third transmitting coil, the second and third transmitting coils being positionable on a different side of a part of the person's ear to the first transmitting coil. In another example, the device is configured to generate and directionally control at least part of the magnetic field by: driving the first transmitting coil and selectively driving the second transmitting coil or the third transmitting coil; or driving the first transmitting coil and differentially driving the second transmitting coil and the third transmitting coil. In another example, the device is configured to differentially drive the second and third transmitting coils by at least one selected from: driving the second and third transmitting coils with respective time-varying electric currents that have different amplitudes; and driving the second and third transmitting coils with respective time-varying electric currents that are out-of-phase from each other. In another example, the device further includes a fourth transmitting coil positionable on a same side of the part of the person's ear as the first transmitting coil. In another example, the device is configured to fixedly couple to the person's ear. In another example, the device comprises a clamp mechanism configured to clamp onto a part of the person's ear. In another example, the clamp mechanism comprises first and second parts positionable on different sides of the part of the person's ear that the clamp is configured to clamp onto, and wherein the first transmitting coil is at least partially provided in the first part. In another example, the device further includes: a control circuit configured to control operations of the drive circuit, wherein the drive circuit is electrically coupled to the first transmitting coil and configured to provide the time-varying electric current to the first transmitting coil. In another example, the control circuit is configured to cause the drive circuit to provide a current pulse or an alternating current (AC) to the first transmitting coil. In another example, the device is configured to generate at least part of the magnetic field by discharging one or more capacitors through the first transmitting coil. In another example, the device is configured to generate magnetic pulses having periods of about 10 milliseconds or less. In another example, the first transmitting coil and the one or more capacitors form at least part of an inductor-capacitor (LC) circuit. In another example, the device further includes: a temperature sensor to measure at least one selected from a temperature of the first transmitting coil and a temperature of a portion of the person's ear proximal to the first transmitting coil; and a control circuit communicatively coupled to the temperature sensor and configured to control operations of the drive circuit, the control circuit being configured to stop the drive circuit from driving of the first transmitting coil in response to a temperature threshold being exceeded. In another example, the temperature threshold is exceeded when the temperature of the portion of the person's ear exceeds 40 degrees centigrade, or when the temperature of the portion of the person's ear increases by more than 2 degrees centigrade. In another example, the temperature threshold is exceeded when the temperature of the first transmitting coil exceeds 40 degrees centigrade, or when the temperature of the first transmitting coil increases by more than 2 degrees centigrade.

In another aspect, the technology relates to a stimulation device, the device being configured to couple to a person's ear and including a first transmitting coil positioned and oriented such that, when the device is coupled to the person's ear, an axis (e.g., a virtual axis) of the first transmitting coil crosses (or intersects) an auricular branch of a Vagus Nerve (ABVN) of the person.

In an example, the device further includes a second transmitting coil positionable on a different side of a part of the person's ear to the first transmitting coil. In another example, the device comprises a clamp mechanism configured to clamp onto a part of the person's ear. In another example, the clamp mechanism comprises first and second parts positionable on different sides of the part of the person's ear that the clamp is configured to clamp onto, and wherein the first transmitting coil is at least partially provided in the first part.

In an example, the method includes generating a magnetic field across the ABVN to activate the ABVN.

In an example, the generating the magnetic field includes driving a first transmitting coil positioned on a first side of a part of a person's ear with a time-varying electric current. In another example, the generating the magnetic field further includes driving a second transmitting coil positioned on a second side of the part of the person's ear different from the first side with a time-varying electric current. In another example, the generating the magnetic field further includes directionally controlling the magnetic field by: selectively driving a second transmitting coil or a third transmitting coil with a time-varying electric current, the second and third transmitting coils being positioned on a second side of the part of the person's ear different from the first side, or differentially driving a second transmitting coil and a third transmitting coil with respective time-varying electric currents, the second and third transmitting coils being positioned on a second side of the part of the person's ear different from the first side. In another example, the second and third transmitting coils are differentially driven by at least one selected from: driving the second and third transmitting coils with respective time-varying electric currents that have different amplitudes; and driving the second and third transmitting coils with respective time-varying electric currents that are out-of-phase from each other. In another example, a ferrite piece is positioned on a second side of the part of the person's ear different from the first side during the generating the magnetic field and such that a magnitude of the magnetic field across the ABVN is increased compared to when the ferrite piece is excluded. In another example, the generating the magnetic field includes driving a first transmitting coil with a time-varying electric current, the first transmitting coil being shaped and positioned such that a first end of the first transmitting coil is on a first side of a part of a person's ear and a second end of the first transmitting coil is on a second side of a part of the person's ear different from the first side. In another example, the driving the first transmitting coil includes providing an electric current pulse to the first transmitting coil or providing an alternating current (AC) to the first transmitting coil. In another example, the method further includes measuring a temperature of a portion of the person's ear proximal to the first transmitting coil and stopping the driving of the first transmitting coil in response to determining that a temperature threshold is exceeded. In another example, the temperature threshold is exceeded when the temperature of the portion of the person's ear exceeds 40 degrees centigrade or when the temperature of the portion of the person's ear increases by more than 2 degrees centigrade. In another example, the method further includes measuring a temperature of the first transmitting coil and stopping the driving of the first transmitting coil when a temperature threshold is exceeded.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

The accompanying drawings, together with the specification, illustrate embodiments of the subject matter of the present disclosure, and, together with the description, serve to explain principles of embodiments of the subject matter of the present disclosure.

FIG. 1 depicts a front view of a person's ear.

FIG. 2 depicts a stimulation device according to some embodiments coupled to the person's ear.

FIG. 3 depicts a partially transparent front view of the stimulation device of FIG. 2.

FIG. 4 depicts the transmitting coils of the stimulation device of FIG. 2.

FIG. 5 depicts a front view of the stimulation device of FIG. 2.

FIG. 6 depicts a partially transparent side view of the stimulation device of FIG. 5.

FIG. 7 depicts a partially transparent side view of another stimulation device according to some embodiments.

FIG. 8 depicts a partially transparent side view of another stimulation device according to some embodiments.

FIG. 9 depicts a partially transparent side view of another stimulation device according to some embodiments.

FIG. 10 depicts another stimulation device according to some embodiments coupled to a person's ear.

FIG. 11 depicts another stimulation device according to some embodiments coupled to a person's ear.

FIG. 12 depicts a partially transparent perspective view of the stimulation device of FIG. 11.

FIG. 13 depicts a method of stimulating the Auricular Branch of the Vagus Nerve according to some embodiments.

FIG. 14 depicts some details of the task of driving the at least one of the one or more transmitting coils of FIG. 13 according to some embodiments.

### DETAILED DESCRIPTION

Hereinafter, example embodiments will be described in more detail with reference to the accompanying drawings, in which like reference numbers refer to like elements throughout. The subject matter of the present disclosure, however, may be embodied in various different forms, and should not be construed as being limited to only the illustrated and described embodiments herein. Rather, these embodiments are provided as examples so that this disclosure will be thorough and complete, and will fully convey the aspects and features of the present disclosure to those skilled in the art. Accordingly, processes, elements, and techniques that are not necessary to those having ordinary skill in the art for a complete understanding of the aspects and features of the present disclosure may not be described. Unless otherwise noted, like reference numerals denote like elements throughout the attached drawings and the written description, and thus, descriptions thereof will not be repeated. In the drawings, the relative sizes of elements, layers, and regions may be exaggerated for clarity.

It will be understood that, although the terms “first,” “second,” “third,” etc., may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present disclosure.

Spatially relative terms, such as “beneath,” “below,” “lower,” “under,” “above,” “upper,” and the like, may be used herein for ease of explanation to describe one element or feature's relationship to another element(s) or feature(s) as illustrated in the figures. It will be understood that the spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented (e.g., rotated 90 degrees or at other orientations) and the spatially relative descriptors used herein should be interpreted accordingly.

It will be understood that when an element or layer is referred to as being “on,” “connected to,” or “coupled to” another element or layer, it can be directly on, connected to, or coupled to the other element or layer, or one or more intervening elements or layers may be present. In addition, it will also be understood that when an element or layer is referred to as being “between” two elements or layers, it can be the only element or layer between the two elements or layers, or one or more intervening elements or layers may also be present.

The terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises,” “comprising,” “includes,” and “including,” when used in this specification, specify the presence of the stated features, integers, acts, operations, elements, and/or components, but do not preclude the presence or addition of one or more other features, integers, acts, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list.

As used herein, the terms “substantially,” “about,” and similar terms are used as terms of approximation and not as terms of degree, and are intended to account for the inherent deviations in measured or calculated values that would be recognized by those of ordinary skill in the art. Further, the use of “may” when describing embodiments of the present disclosure refers to “one or more embodiments of the present disclosure.” As used herein, the terms “use,” “using,” and “used” may be considered synonymous with the terms “utilize,” “utilizing,” and “utilized,” respectively.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The electronic or electric devices and/or any other relevant devices or components according to embodiments of the present disclosure described herein may be implemented utilizing any suitable hardware, firmware (e.g. an application-specific integrated circuit), software, or a combination of software, firmware, and hardware. For example, the various components of these devices may be formed on one integrated circuit (IC) chip or on separate IC chips. Further, the various components of these devices may be implemented on a flexible printed circuit film, a tape carrier package (TCP), a printed circuit board (PCB), or formed on one substrate. Further, the various components of these devices may be a process or thread, running on one or more processors, in one or more computing devices, executing computer program instructions and interacting with other system components for performing the various functionalities described herein. The computer program instructions may be stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the spirit and scope of the example embodiments of the present disclosure.

Unless otherwise defined, all terms (including technical and scientific terms) used herein have the same meaning as commonly understood by one of ordinary skill in the art to which the present disclosure belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

As explained above, the Vagus Nerve (VN) plays a role in controlling various involuntary functions of the body, and it may be desirable to controllably activate the VN by providing electrical stimulation to the VN to improve the performance of some of these functions.

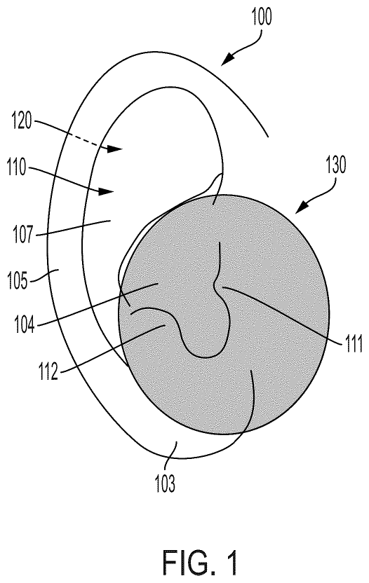

The VN extends through the torso of the body and through the right and left sides of the head proximal to the ears. The Auricular Branch of the VN (ABVN) is a portion of the VN that branches into the ears. FIG. 1 depicts a front view of a person's ear. The ear 100 may have a front side 110, which is depicted in FIG. 1, and a back side 120 opposite to the front side 110. The ear 100 may include the tragus 111, antitragus 112, lobule 103, concha 104, antihelix 107, and helix 105. The ABVN may extend within a region 130 of the ear 100 that may include, for example, the tragus 111, antitragus 112, concha 104, and lobule 103. Although any portion of the VN may be stimulated, it is desirable to stimulate the ABVN because most portions of the VN are deep inside the body except for at a few places, such as portions of the VN in the neck and ears. It is also useful to stimulate the ABVN because it is isolated from other nerves and organs of the body, stimulation of which may not be desired. For example, the portion of the VN in the neck is near the phrenetic nerve, which plays a role in controlling the person's diaphragm while breathing, and thus, may not be desirable to stimulate together with the VN.

The VN may be stimulated by an implanted device having an exposed electrode that is in contact with, or in close proximity to, the VN and configured to provide an electric current to the VN. However, it is desirable to be able to provide stimulation to the VN without the bodily invasion required of an implantable device and the added complexities of powering the implantable device when it is within the body.

The VN may be stimulated by applying, via an electrode, an electric current to a portion of the person's ear near the ABVN such that the electric current is provided to the ABVN through the skin and any other intervening tissue. However, the impedance between the electrode and the ABVN is difficult to control and to accurately predict, which makes this manner of stimulating the VN unreliable and difficult to control. For example, the ear generally has an odd geometry that differs from person to person. Therefore, it is difficult to provide a uniformly shaped product that is able to reliably make and maintain suitable contact between the electrode and the ear (e.g., without an air gap forming therebetween) for consumers. As another example, moisture on a person's ear, which affects the impedance between the electrode and the ABVN, may vary unpredictably based on the temperature of the person's environment, the person's level of physical activity, and the person's mood, among other factors.

Devices according to some embodiments disclosed herein are configured to stimulate the ABVN by generating a time-varying magnetic field that provides a time-varying magnetic flux over the ABVN sufficient to activate the ABVN (e.g., to generate or induce an action potential in the ABVN). The time-varying magnetic field generates electric currents in the ABVN according to Maxwell's equations, and thus, the magnetic field can be generated such that the time-varying magnetic flux over the ABVN is sufficiently large to activate the ABVN. Devices that activate that ABVN by generating such magnetic fields over the ABVN provide a non-invasive manner to activate the VN that is substantially unaffected by the electrical impedance between the device (e.g., a coil in the device) and the ABVN. Devices and methods of the present disclosure will now be discussed in more detail.

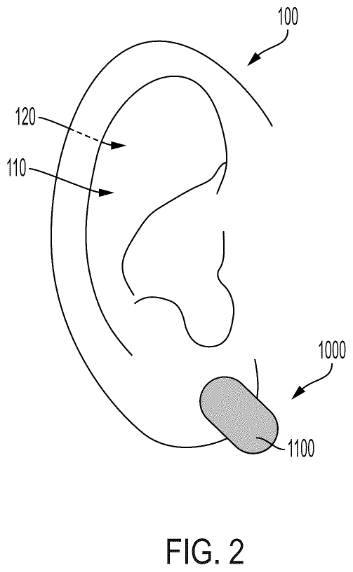

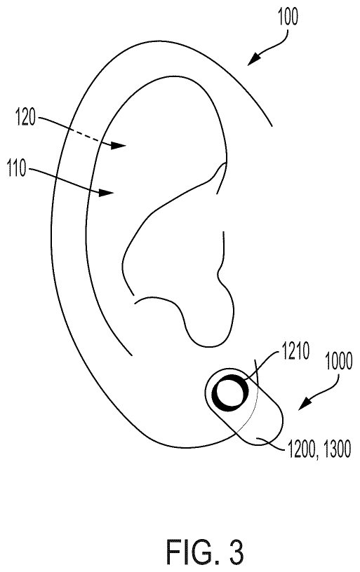

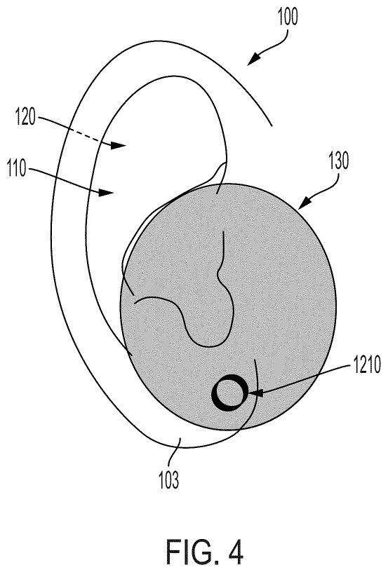

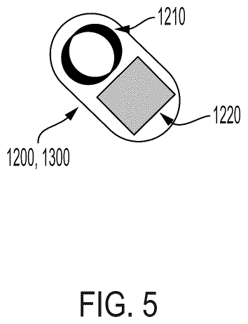

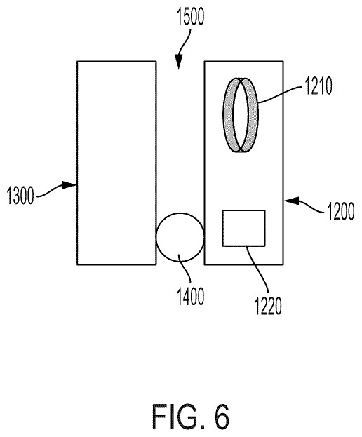

FIG. 2 depicts a stimulation device 1000 according to some embodiments coupled to the person's ear 100. FIG. 3 depicts a partially transparent view of the stimulation device 1000 of FIG. 2. FIG. 4 depicts the transmitting coil of the stimulation device 1000 of FIG. 2. FIG. 5 depicts a front view of the stimulation device 1000 of FIG. 2. FIG. 6 depicts a cross-sectional view along line 6-6′ in FIG. 5 of the stimulation device 1000 of FIG. 2.

Referring concurrently to FIGS. 2-6, the stimulation device 1000 may include a first part 1200 and a second part 1300 coupled to the first part 1200. The first part 1200 may include a first transmitting coil 1210 and a first control circuit 1220. The first part 1200 may include a first drive circuit electrically coupled to the first transmitting coil 1210 and to the first control circuit 1220, and the first drive circuit may be configured to drive the first transmitting coil 1210, for example, under the control of the first control circuit 1220. In some embodiments, the first part 1200 may include a temperature sensor configured to measure at least one selected from a temperature of a portion of the ear 100 in proximity to (e.g., adjacent to or beneath) the first transmitting coil 1210 when the device 1000 is coupled to the ear 100 and a temperature of the first transmitting coil 1210. The first temperature sensor may be on a skin-contacting surface of the device 1000.

The first control circuit 1220 may be configured to control at least some operations of the device 1000 and may include, for example, an integrated chip or a printed circuit board. The first control circuit 1220 may be configured to control operations of the first drive circuit and operations of the first temperature sensor.

The first and second parts 1200 and 1300 may be coupled or couplable together, for example, by a connector device 1400, and may be fixed relative to each other or adjustable relative to each other. In some embodiments, the connector device 1400 is a component fixedly connecting the first and second parts 1200 and 1300 together such that their positions and orientations relative to each other are fixed. For example, the connector device 1400 may include a rigid structure bridging between, and fixedly attached to, the first and second parts 1200 and 1300. In some other embodiments, the connector device 1400 is configured to allow the first and second parts 1200 and 1300 to move relative to each other. For example, the connector device 1400 may include a rotation hinge configured such that the first and second parts 1200 and 1300 are rotatable relative to each other at a rotation axis, which may extend in and out of the page as shown in the cross-sectional view shown in FIG. 6. In some other embodiments, the connector device 1400 includes a linearly translatable connector coupled between the first and second parts 1200 and 1300 and configured such that the first and second parts 1200 and 1300 are linearly translatable relative to each other, for example, along a direction parallel to one or both of the first and second transmitting coils 1210 and 1310. In some embodiments, the linearly translatable connector may utilize telescoping technology, variations thereof, or other technology configured to allow for linear translation. The connector device 1400 may be configured such that the orientations of the first and second parts 1200 and 1300 relative to each other remain fixed when the first and second parts 1200 and 1300 linearly translate relative to each other.

The first and second parts 1200 and 1300 may be positionable on different (e.g., opposite) sides of a part of the ear 100 when the device 1000 is coupled to the ear 100. For example, the first and second parts 1200 and 1300 may be respectively positionable on the front and back sides 110 and 120 of the ear 100, such as front and back side portions of the lobule 103, respectively, of the ear 100. A space 1500 (e.g., a gap) may be provided between the first and second parts 1200 and 1300 to receive and accommodate the part of the ear 100 to be positioned between the first and second parts 1200 and 1300 when the device 1000 is coupled to the ear 100. The device 1000 may include an outer casing 1100 to house the first and second parts 1200 and 1300 and the connector device 1400. The outer casing 1100 may be substantially formfitting around at least part of the first and second parts 1200 and 1300 and the connector device 1400 such that the space 1500 is not closed by the outer casing 1100.

The device 1000 may be couplable to the ear 100 by any suitable means. Coupling the device 1000 to the ear 100 allows for hand-free use of the device 1000, which may be desirable when the device 1000 needs to be used for long periods of time during the day or throughout the night. Coupling the device 1000 to the ear 100 may also allow the position and orientation of the first transmitting coil 1210 relative to a set part of the ear 100 to be fixed, which may allow the device 1000 to reliably stimulate the ABVN. In some embodiments, when the connector device 1400 includes the rotation hinge or the linearly translatable component, the first and second parts 1200 and 1300 may be movable towards and away from each other so that the first and second parts 1200 and 1300 can be clamped down on the part of the ear 100 to couple the device 1000 to the ear 100. For example, the first and second parts 1200 and 1300 and the connector device 1400 may form at least some parts of a clamp mechanism configured to clamp the device 1000 onto the ear 100.

However, the present disclosure is not limited thereto, and the device 1000 may be couplable to the ear 100 by any suitable means. For example, the device 1000 may include a separate clamp mechanism configured to fixedly clamp the device 1000 onto the ear 100. In some embodiments, the second part 1300 and the connector device 1400 are excluded. The separate clamp mechanism may include a first part, a second part, and/or a connector, where the first and second parts of the separate clamp mechanism are movable (e.g., rotatable and/or linearly translatable) relative to each other, for example, via the connector of the separate clamp mechanism. The separate clamp mechanism may be configured to clamp the first and second parts onto different (e.g., opposite) sides of at least a part of the ear 100.

In some examples, the device 1000 may include a structural loop configured (e.g., shaped and/or sized) to at least partially encircle the base of the ear 100 where the ear 100 attaches to the head to couple the device 1000 to the ear 100. In some examples, the device 1000 may include a structural anchor piece configured (e.g., shaped and/or sized) to be fitted at least partially into the concha 104 and/or an ear canal of the ear 100 to at least partially stabilize the position and/or orientation of the device 1000. The coupling mechanisms described herein for coupling the device 1000 to the ear 100 may be used individually or in any suitable combination.

The device 1000 may be configured such that, when the device 1000 is coupled to the ear 100, an axis (e.g., a virtual axis) of the first transmitting coil 1210 intersects the ABVN. For example, the first and second parts 1200 and 1300 of the device 1000 may be shaped and sized, and the first transmitting coil 1210 may be positioned and oriented, such that the device 1000 is couplable to the ear 100 so that the virtual axis of the first transmitting coil 1210 crosses (or intersects) the ABVN. In some embodiments, when the first transmitting coil 1210 is a straight solenoid coil, the axis of the first transmitting coil 1210 is a straight axis line (e.g., a virtual straight line) extending through the respective centers of the two ends of the solenoid coil. In some embodiments, when the first transmitting coil 1210 is a curved solenoid coil, the virtual axis may be a curved line subject to parameters that the curved line extends through the respective centers of the two ends of the solenoid coil, a middle portion of the curved line extends (e.g., extends substantially through the solenoid coil) between the respective centers of the two ends, and outer portions of the curved line extending outwards from the respective centers of the two ends away from the solenoid coil may be straight portions extending in directions respectively normal (e.g., perpendicular) to disks (e.g., virtual disks) having perimeters defined by the two ends of the solenoid coil.

The first transmitting coil 1210 may be configured such that, when the device 1000 is in proximity to the ear 100 (e.g., coupled to the ear 100) and the first transmitting coil 1210 is driven by the first drive circuit with a time-varying electric current, the first transmitting coil 1210 may generate at least part of (e.g., part or all) a magnetic field that provides a time-varying magnetic flux across the ABVN sufficient to activate the ABVN by inducing sufficiently large electric currents in the ABVN. The magnitude of electric currents induced in the ABVN is proportional to the magnitude of the time-rate of change of the magnetic field across the ABVN in accordance with Maxwell's equations, and the time-rate of change of the magnetic field across the ABVN depends in part on the magnitude of the magnetic field that the first transmitting coil 1210 can generate in response to a time-varying electric current providable from the first drive circuit. Accordingly, in some embodiments, the number of loops of a wire of the first transmitting coil 1210, a material of the wire, a diameter of the first transmitting coil 1210, and/or the presence of and type of material (e.g., ferrite) of a core of the first transmitting coil 1210 may be set such that, when the first transmitting coil 1210 is driven with a time-varying electric current providable by the first drive circuit, the magnetic field provides the time-varying magnetic flux across the ABVN sufficient to activate the ABVN. The time-varying electric currents providable by the first drive circuit (e.g., via control of the first control circuit 1220) may include a current pulse having a time duration within a set range and a maximum magnitude within a set range and/or an alternating current (AC) having frequencies within a set range and amplitudes within a set range. In some embodiments, the time duration may include, for example, about 100 ms or less, about 50 ms or less, about 20 ms or less, about 10 ms or less, or about 5 ms or less. Short (e.g., temporally short) electric current pulses in the first transmitting coil 1210 may generate short magnetic pulses through the ABVN that induce short electric current pulses in the ABVN.

The first drive circuit may include one or more capacitors electrically coupled to the wire of the first transmitting coil 1210. The first transmitting coil 1210 may be driven with the time-varying electric current by discharging at least one of the one or more capacitors through the first transmitting coil 1210. The capacitances of the one or more capacitors may be set to control or set the rate of discharge of the at least one of the one or more capacitors through the first transmitting coil 1210. In some embodiments, an inductor-capacitor (LC) circuit may be formed at least in part by the wire of the first transmitting coil 1210 and the one or more capacitors of the first drive circuit. The LC circuit may be configured so that energy discharged from a first capacitor of the one or more capacitors through the wire of the first transmitting coil 1210 is recaptured from the first transmitting coil 1210 by the first capacitor and/or by a second capacitor of the one or more capacitors of the first driving circuit. The LC circuit may be configured to controllably allow energy to resonate between the wire of the first transmitting coil 1210 and the one or more capacitors of the first driving circuit for a set period of time. A transistor (e.g., a field-effect transistor (FET)) may be included and configured to break the LC circuit when a set portion (e.g., substantially all) of the energy resonating in the LC circuit is stored in the one or more capacitors of the first driving circuit in order to end the resonance. A switched inductor may be included and configured to charge at least one capacitor of the one or more capacitors of the first driving circuit up to a set initial voltage.

The first temperature sensor may be communicatively coupled to the control circuit 1220 and may be configured to measure one or both of a temperature of the first transmitting coil 1210 and a temperature of a portion of the ear 100 proximal to the first transmitting coil 1210 when the device 1000 is coupled to the ear 100. In some embodiments, the first temperature sensor may be positioned at a side of the first part 1200 facing the space 1500, for example, between the space 1500 and the first transmitting coil 1210. Measuring the temperature of at least one selected from the portion of the ear 100 and the first transmitting coil 1210 may allow the control circuit 1220 to stop the first drive circuit from driving the first transmitting coil 1210 once a temperature threshold is exceeded.

In some embodiments, the first temperature sensor may be positioned in the first part 1200 proximal to the portion of the ear 100 and/or proximal to the first transmitting coil 1210. For example, the first temperature sensor may be positioned between the first transmitting coil 1210 and the space 1500. The first temperature sensor may include, for example, a resistance temperature detector (RTD), a semiconductor temperature sensor, a thermocouple, and/or a thermistor.

The temperature threshold may be exceeded, for example, when the temperature of the portion of the ear 100 exceeds a set temperature threshold (e.g., a temperature within the range of about 38 to about 42 degrees centigrade, such as about 40 degrees centigrade), when the temperature of the portion of the ear 100 increases by more than a set temperature increase (e.g., a temperature increase within the range of about 1 to about 4 degrees centigrade, such as about 2 degrees centigrade) compared to a time before the first drive circuit began to drive the first transmitting coil 1210, when the temperature of the first transmitting coil 1210 exceeds a set temperature threshold (e.g., a temperature within the range of about 38 to about 42 degrees centigrade, such as about 40 degrees centigrade), and/or when the temperature of first transmitting coil 1210 increases by more than a set temperature increase (e.g., a temperature increase within the range of about 1 to about 4 degrees centigrade, such as about 2 degrees centigrade) compared to a time before the first drive circuit began to drive the first transmitting coil 1210.

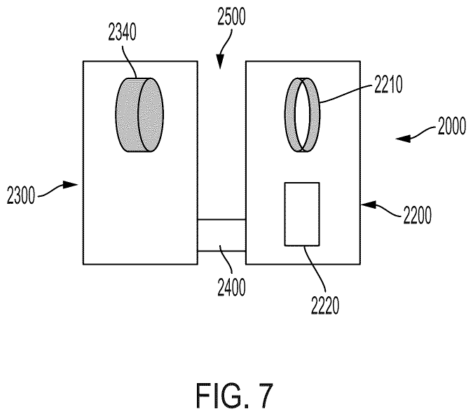

FIG. 7 depicts a cross-sectional view of another stimulation device 2000 according to some embodiments. The device 2000 may include features similar to the features of other stimulation devices illustrated and described herein or otherwise within the scope of the present disclosure, and differences will be primarily be described.

The device 2000 includes a first part 2200 including a first transmitting coil 2210 and a first control circuit 2220, a second part 2300 including a magnetic material 2340 (e.g., a magnetically soft material, a ferromagnetic or ferrimagnetic material, etc., such as a ferrite piece), a space 2500 between the first and second parts 2200 and 2300, and a connector device 2400 configured to couple the first and second parts 2200 and 2300 together.

The magnetic material 2340 will tend to guide the magnetic field generated by the first transmitting coil 2210 toward and through itself, thereby increasing a magnitude (e.g., a maximum magnitude) of the magnetic field between the first transmitting coil 2210 and the magnetic material 2340 compared to if the magnetic material 2340 were excluded. The magnetic material 2340 may be positioned in the second part 2300 such that the first transmitting coil 2210 and the magnetic material 2340 are positionable on different sides of the part of the ear 100 that the device 2000 is couplable to and such that a magnitude (e.g., a maximum magnitude) of the magnetic field across the ABVN is greater than if the magnetic material 2340 were excluded. For example, the first transmitting coil 2210 and the magnetic material 2340 may be positionable such that a part of the ABVN is between the first transmitting coil 2210 and the magnetic material 2340. In some embodiments, an axis (e.g., a virtual axis) of the first transmitting coil 2210 may cross or intersect the magnetic material 2340, or the first transmitting coil 2210 may be positionable and orientable such that its virtual axis crosses (or intersects) the magnetic material 2340.

The magnetic material 2340 may have any suitable shape and size. The magnetic material 2340 is illustrated in FIG. 7 as having a cylindrical shape having a diameter substantially the same as the diameter of the first transmitting coil 2210, but the present disclosure is not limited thereto. For example, the diameter of the first magnetic material 2340 may be smaller than the diameter of the first transmitting coil 2210 in order to focus the magnetic field generated by the first transmitting coil 2210. In some embodiments, the magnetic material 2340 may be shaped such that, when the device 2000 is coupled to a part of the ear 100 with the first transmitting coil 2210 on a first side of the part of the ear 100 and the magnetic material 2340 on a second side of the part of the ear 100 different from the first side, the magnetic material 2340 extends around the part of the ear 100 from the second side to the first side. For example, the magnetic material 2340 may extend through or along side the connector device 2400 within the device 2000.

In some embodiments, the magnetic material 2340 is a first magnetic material, and a second magnetic material (e.g., second ferrite piece) is included in the first part 2200. In some other embodiments, the second magnetic material is included in the first part 2200 and the first magnetic material 2340 is excluded. The geometry of the magnetic field generated by the first transmitting coil 2210 is affected by the presence, position, and shape of magnetic materials, and thus, the first and/or second magnetic materials may be shaped, sized, positioned, and/or oriented to control the shape of the magnetic field generated by the first transmitting coil 2210.

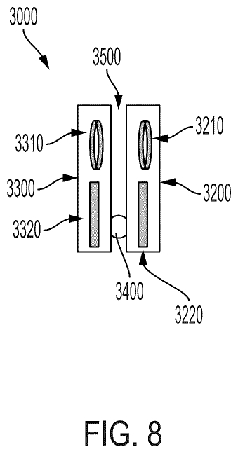

FIG. 8 depicts a cross-sectional view of another stimulation device 3000 according to some embodiments. The device 3000 may include features similar to the features of other stimulation devices illustrated and described herein or otherwise within the scope of the present disclosure, and differences will be primarily be described.

The device 3000 includes a first part 3200 including a first transmitting coil 3210 and a first control circuit 3220, a second part 3300 including a second transmitting coil 3310 and a second control circuit 3320, a space 3500 between the first and second parts 3200 and 3300, and a connector device 3400 configured to couple the first and second parts 3200 and 3300 together.

The second part 3300 may include a second temperature sensor and a second driving circuit configured to provide a time-varying electric current to the second transmitting coil 3310. The second control circuit 3320 may be configured to control the operations of the second driving circuit and the second temperature sensor. The second transmitting coil 3310, the second driving circuit, the second temperature sensor, and the second control circuit 3320 may have any features that the first transmitting coil 3210, the first driving circuit, the first temperature sensor, and the first control circuit 3220 may have, respectively. In some embodiments, the second control circuit 3320 is omitted and the second transmitting coil 3310, the second driving circuit, and the second temperature sensor may be controllable by the first control circuit 3220. In some other embodiments, more than two control circuits are provided to control the first and second transmitting coils 3210 and 3310, the first and second driving circuits, and the first and second temperature sensors.

The second transmitting coil 3310 may be positioned in the second part 3300 such that the first transmitting coil 3210 and the second transmitting coil 3310 are positionable on different sides of the part of the ear 100 that the device 3000 is couplable to, and the first and second transmitting coils 3210 and 3310 can be driven together (e.g., concurrently or simultaneously) in order to generate at least part of a magnetic field across the ABVN that is greater than if only one of the first transmitting coil 3210 and the second transmitting coil 3310 are driven. For example, the first transmitting coil 3210 and the second transmitting coil 3310 may be positionable such that a part of the ABVN is between the first transmitting coil 3210 and the second transmitting coil 3310.

When a single transmitting coil is driven, the magnetic field lines of the magnetic field generated by the transmitting coil form closed loops extending through the transmitting coil, out one end of the transmitting coil, and around the transmitting coil to the other end of the transmitting coil to complete the loop. Thus, the magnetic field spreads out from the one end of the transmitting coil, undermining the transmitting coil's ability to direct the magnetic field through the ABVN. When the first and second transmitting coils 3210 and 3310 are placed in proximity to each other and are generally aligned with each other, and when the first and second transmitting coils 3210 and 3310 are driven together, the magnetic field generated by one of the first and second transmitting coils 3210 and 3310 is straightened out in the region between the first and second transmitting coils 3210 and 3310 by the presence of the other one of the first and second transmitting coils 3210 and 3310.

In some embodiments, the first and second transmitting coils 3210 and 3310 may be positioned and oriented such that their axes (e.g., virtual axes) are aligned, or the first and second transmitting coils 3210 and 3310 may be positionable and orientable such that their virtual axes are align-able. In some embodiments, the first and second transmitting coils 3210 and 3310 may be positioned and oriented such that their virtual axes intersect to form an angle within a range of about 90 degrees to about 180 degrees, or the first and second transmitting coils 3210 and 3310 may be positionable and orientable such that their virtual axes are crossable (or intersect-able) to form an angle within a range of about 90 degrees to about 180 degrees. The position and orientation of the first and second transmitting coils 3210 and 3310 relative to each other may be adjustable, for example, by the adjustability of the first and second parts 3200 and 3300, such as the when the connector device 1400 is a hinge allowing the first and second parts 3200 and 3300 to be rotated relative to each other. The magnetic field generated by the device 3000 may therefore be directionally controlled by controlling the relative position and/or orientation of the first and second transmitting coils relative to each other.

The first and second transmitting coils 3210 and 3310 may be driven together. For example, when the first and second transmitting coils 3210 and 3310 are driven with AC current, they may be driven in phase. When the first and second transmitting coils 3210 and 3310 are driven with electric current pulses, the pulses may be provided substantially concurrently (e.g., simultaneously).

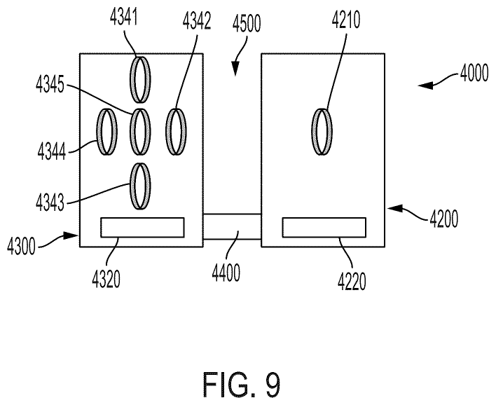

FIG. 9 depicts a cross-sectional view of another stimulation device 4000 according to some embodiments. The device 4000 may include features similar to the features of other stimulation devices illustrated and described herein or otherwise within the scope of the present disclosure, and differences will be primarily be described.

The device 4000 may include a first part 4200 including a first transmitting coil 4210 and a first control circuit 4220, a second part 4300 including a plurality of second transmitting coils and a second control circuit 4320, a space 4500 between the first and second parts 4200 and 4300, and a connector device 4400 configured to couple the first and second parts 4200 and 4300 together.

The plurality of second transmitting coils of the second part 4300 may include a first-second transmitting coil 4341, a second-second transmitting coil 4342, a third-second transmitting coil 4343, a fourth-second transmitting coil 4344, and a fifth-second transmitting coil 4345. However, the present disclosure is not limited thereto, and the plurality of transmitting coils of the second part 4300 may be two transmitting coils, three transmitting coils, four transmitting coils, or six or more transmitting coils. In some embodiments, virtual axes of the first transmitting coil 4210 and the fifth-second transmitting coil 4345 may be aligned or align-able, and the first to fourth-second transmitting coils 4341, 4342, 4343, and 4344 may be arranged in the second part 4300 to be around the fifth-second transmitting coil 4345.

The second part 4300 may include a second driving circuit configured to selectively and/or differentially drive the first to fifth-second transmitting coils 4341, 4342, 4343, 4344, and 4345. For example, the second driving circuit may be configured to selectively drive one or more of the first to fifth-second transmitting coils 4341, 4342, 4343, 4344, and 4345 and to not drive the remaining transmitting coils of the first to fifth-second transmitting coils 4341, 4342, 4343, 4344, and 4345. In some embodiments, the second driving circuit may be configured to differentially drive first to fifth-second transmitting coils 4341, 4342, 4343, 4344, and 4345 or the transmitting coils selected from among the first to fifth-second transmitting coils 4341, 4342, 4343, 4344, and 4345 that are selected to be driven. Differentially driving some or all of the first to fifth-second transmitting coils 4341, 4342, 4343, 4344, and 4345 may include driving the transmitting coils with respective time-varying electric currents having different amplitudes and/or driving the transmitting coils with respective time-varying electric currents that are controllably out-of-phase from each other.

The device 4000 is therefore configured to generate and directionally control at least part of a magnetic field by selectively and/or differentially driving the first to fifth-second transmitting coils 4341, 4342, 4343, 4344, and 4345, alone or together with driving the first transmitting coil 4210.

In some embodiments, the first part 4200 may also include a plurality of first transmitting coils having any number of the first transmitting coils, any arrangement of the first transmitting coils, and any configuration of the first transmitting coils that the second part 4300 may have, and the pluralities of first and second transmitting coils of the first and second parts 4200 and 4300 may be the same or different in each of the number of the transmitting coils, the arrangement of the transmitting coils, and configuration of the transmitting coils. For example, the first part 4200 may include five transmitting coils having virtual axes aligned or alignable with respective virtual axes of the first to fifth-second transmitting coils 4341, 4342, 4343, 4344, and 4345 of the second part 4300, and the five transmitting coils of the first part 4200 may be configured to be selectively and/or differentially driven by a first driving circuit of the first part 4200. In some other embodiments, the first part 4200 may include only three transmitting coils.

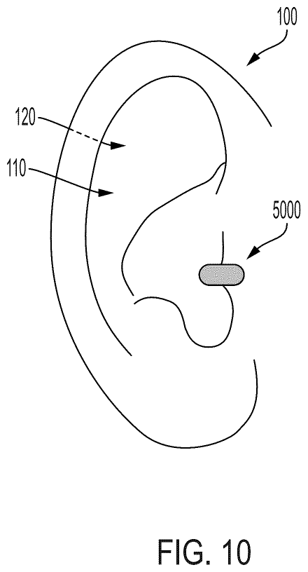

FIG. 10 depicts another stimulation device 5000 according to some embodiments coupled to a person's ear 100. The device 5000 may include features similar to the features of other stimulation devices illustrated and described herein or otherwise within the scope of the present disclosure, and differences will be primarily be described. The device 5000 may be configured (e.g., shaped and sized) to couple to or to be positioned on the tragus 111. For example, the device 5000 may have a first part and a second part positionable on different sides of the tragus 111.

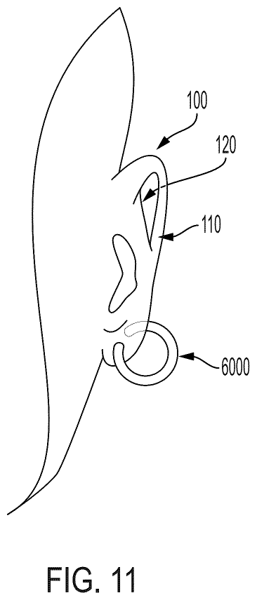

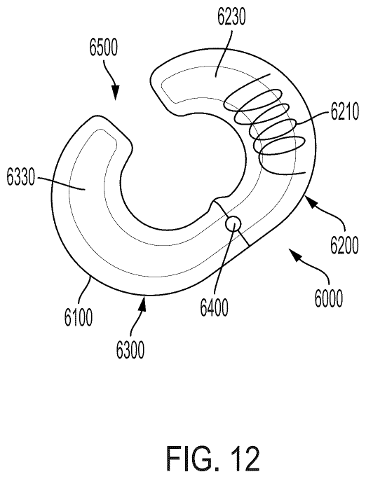

FIG. 11 depicts another stimulation device 6000 according to some embodiments coupled to a person's ear 100. FIG. 12 depicts a perspective view of the stimulation device 6000 of FIG. 11. The device 6000 may include features similar to the features of other stimulation devices illustrated and described herein or otherwise within the scope of the present disclosure, and differences will be primarily be described.

The device 6000 includes a first part 6200 including a first magnetic piece 6230 and at least part of a first transmitting coil 6210, a second part 6300 including a second magnetic piece 6330, a space 6500 between the first and second parts 6200 and 6300, and a connector device 6400 configured to couple the first and second parts 6200 and 6300 together at first ends of the first and second parts 6200 and 6300. The first and second parts 6200 and 6300 may form an open-ringed shape or a horseshoe shape. The connector device 6400 may be, for example, a hinge or linearly translatable connector configured to allow the first and second parts 6200 and 6300 to move toward and away from each other to narrow and broaden the space 6500 in order to allow the device 6000 to be coupled to (e.g., to clamp onto) a part of the ear 100.

The first and second magnetic pieces 6230 and 6330 may extend at least partially through the first and second parts 6200 and 6300, respectively, and may be integrally joined or separated at the first ends of the first and second parts 6200 and 6300. The first transmitting coil 6210 may be configured to be driven by a first driving circuit to generate a magnetic field. The first and second magnetic pieces 6230 and 6330 may include a ferromagnetic or a ferrimagnetic material, such as a ferrite. The first and second magnetic pieces 6230 and 6330 will tend to guide the magnetic field generated by the first transmitting coil 6210, and thus, the first and second magnetic pieces 6230 and 6330 may extend to second ends of the first and second parts 6200 and 6300 adjacent to the space 6500 in order to guide the magnetic field through the part of the ear 100 that occupies the space 6500 when the device 6000 is coupled to the ear 100.

The first transmitting coil 6210 may be positioned along the first part 6200 and may extend at least part way along the first part 6200. At least part of the first magnetic piece 6230 may form a core of the first transmitting coil 6210. In some embodiments, a second transmitting coil may be positioned along the second part 6300 and may extend at least part way along the second part 6300. At least part of the second magnetic piece 6330 may form a core of the second transmitting coil. In some other embodiments, the first transmitting coil 6210 may extend along at least part of both the first and second parts 6200 and 6300. In some such embodiments, the first transmitting coil 6210 may have an open-ringed shape or a horseshoe shape, and the first transmitting coil 6210 may be shaped and sized such that two ends of the first transmitting coil 6210 are positionable on different sides of a part of the ear 100 that the device 6000 is couplable to.

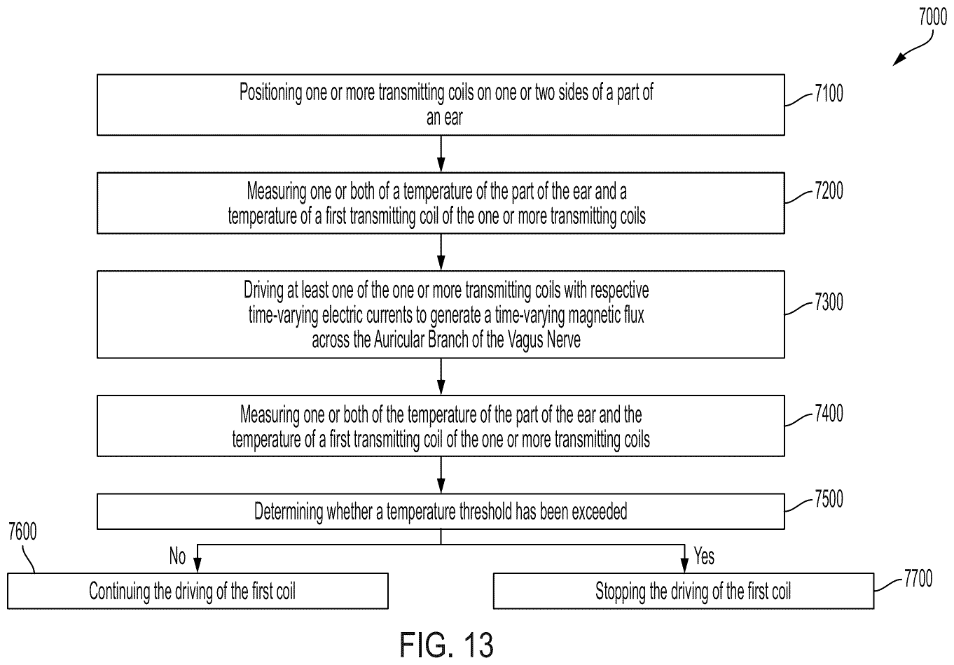

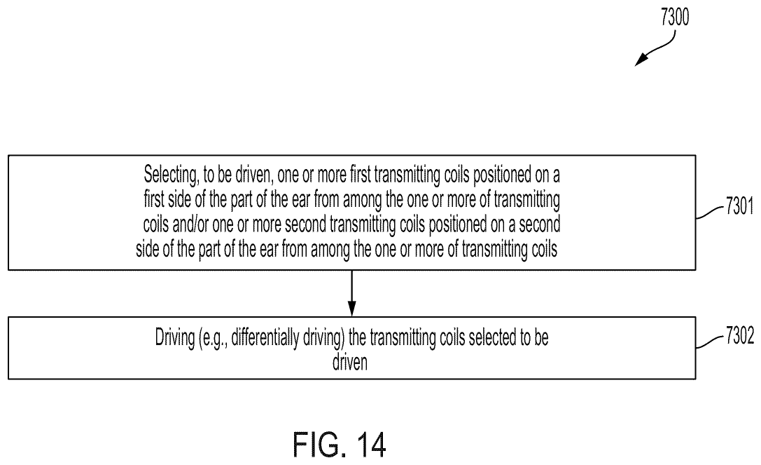

FIG. 13 depicts a method 7000 of stimulating the Auricular Branch of the Vagus Nerve according to some embodiments. FIG. 14 depicts some details of the third task 7300 of driving the at least one of the one or more transmitting coils of the method of FIG. 13 according to some embodiments. The method 7000 may be performed utilizing a stimulation device, including any stimulation device illustrated and described herein or otherwise within the scope or the present disclosure.

Referring to FIG. 13, the method 7000 may include a first task 7100 of positioning one or more transmitting coils on one or two sides of a part of an ear (e.g., the lobule, tragus, antitragus, or concha of the ear). For example, a first transmitting coil may be placed on a first side of the part of the ear, or one or more first transmitting coils may be placed on the first side of the part of the ear and one or more second transmitting coils may be placed on a second side of the part of the ear.

The method 7000 may include a second task 7200 of measuring one or both of a temperature of the part of the ear and a temperature of a first transmitting coil of the one or more transmitting coils. This temperature may be measured before the one or more transmitting coils are driven and may be taken after a set amount of time has passed after the first task 7100 in order to allow the temperature of the first transmitting coil to adjust to a stable temperature after being positioned on the part of the ear.

The method 7000 may include a third task 7300 of driving at least one of the one or more transmitting coils with respective time-varying electric currents to generate a time-varying magnetic flux across the ABVN sufficient to activate the ABVN. The third task 7300 will be described in more detail below with reference to FIG. 14.

The method 7000 may include a fourth task 7400 of measuring one or both of a temperature of the part of the ear and a temperature of the first transmitting coil of the one or more transmitting coils. This temperature may be measured at a set time after the one or more transmitting coils begin to be driven.

The method 7000 may include a fifth task 7500 of determining whether a temperature threshold has been exceeded. This determination may be based on the temperature measured in the fourth task 7400 and, in some embodiments, may also be based on the temperature measured in the second task 7200. For example, the temperature threshold may be exceeded when the temperature of the part of the ear measured in the fourth task 7400 exceeds a set temperature threshold (e.g., a temperature within the range of about 38 to about 42 degrees centigrade, such as about degrees centigrade), when a difference a difference between the temperature of the part of the ear measured in the fourth task 7400 and the temperature of the part of the ear measured in the second task 7200 is greater than a set temperature increase (e.g., a temperature increase within the range of about 1 to about 4 degrees centigrade, such as about 2 degrees centigrade), when the temperature of the first transmitting coil measured in the fourth task 7400 exceeds a set temperature threshold (e.g., a temperature within the range of about 38 to about 42 degrees centigrade, such as about degrees centigrade), and/or when a difference between the temperature of the first transmitting coil measured in the fourth task 7400 and the temperature of the first transmitting coil measured in the second task 7200 is greater than a set temperature increase (e.g., a temperature increase within the range of about 1 to about 4 degrees centigrade, such as about 2 degrees centigrade).

If the temperature threshold is determined in the fifth task 7500 to have not been exceeded, then the first transmitting coil may continue to be driven in a sixth task 7600. However, if the temperature threshold is determined in the fifth task 7500 to have been exceeded, then the driving of the first transmitting coil may be stopped in a seventh task 7700. In some embodiments, in the seventh task 7700, all of the one or more transmitting coils may be stopped in response to the temperature threshold being determined in the fifth task 7500 to have been exceeded. Stopping the driving of some or all of the one or more transmitting coils may be desirable to prevent overheating or injury to the person (e.g., the person's ear) and/or overheating, damage, or malfunction of the device.

Referring to FIG. 14, the third task 7300 may include a first subtask 7301 of selecting, to be driven, one or more first transmitting coils positioned on a first side of the part of the ear from among the one or more transmitting coils and/or one or more second transmitting coils positioned on a second side of the part of the ear from among the one or more transmitting coils. For example, one first transmitting coil positioned on the first side of the part of the ear may be selected to be driven, and no second transmitting coils positioned on the second side of the part of the ear may be selected to be driven. As another example, two or more first transmitting coils positioned on the first side of the part of the ear may be selected to be driven, and no second transmitting coils positioned on the second side of the part of the ear may be selected to be driven. Driving a plurality of transmitting coils on only one side of the part of the ear may allow a shape of the magnetic field across the ABVN to be controlled in ways that may be difficult or impossible to do by driving only a single transmitting coil or by driving transmitting coils on both the first and second sides of the part of the ear. In another example, one first transmitting coil positioned on the first side of the part of the ear, and one or more second transmitting coils positioned on the second side of the part of the ear, may be selected to be driven. In another example, a plurality of first transmitting coils positioned on the first side of the part of the ear, and a plurality of second transmitting coils positioned on the second side of the part of the ear, may be selected to be driven.

The third task 7300 may include a second subtask 7302 of driving (e.g., differentially driving) the transmitting coils selected to be driven in the first subtask 7301. For example, all of the selected transmitting coils may be driven together in-phase and their respective time-varying electric currents may have substantially identical amplitudes. In some embodiments, the selected transmitting coils may be differentially driven, for example, by driving the selected transmitting coils with different amplitudes and/or by driving the selected transmitting coils out-of-phase and controlling the phase difference between respective time-varying electric currents of the selected transmitting coils. For example, a first transmitting coil positioned on the first side of the part of the ear, and a second transmitting coil and a third transmitting coil positioned on the second side of the part of the ear, may be selected to be driven, and the second and third transmitting coils may be driven with respective time-varying electric currents having different amplitudes and/or with a phase difference between the respective time-varying electric currents. In some embodiments, the time-varying electric current of the first transmitting coil may have an amplitude that differs from at least one selected from the respective time-varying electric currents of the second and third transmitting coils and/or may have a controlled phase difference relative to at least one selected from the respective time-varying electric currents of the second and third transmitting coils.

While this invention has been described in detail with particular references to embodiments thereof, the embodiments described herein are not intended to be exhaustive or to limit the scope of the invention to the exact forms disclosed. Persons skilled in the art and technology to which this invention pertains will appreciate that alterations and changes in the described structures and methods of assembly and operation can be practiced without meaningfully departing from the principles, spirit, and scope of this invention, as set forth in the following claims and equivalents thereof.

What is claimed is:

1. A stimulation device, the device comprising:
a first transmitting coil; and
a drive circuit electrically coupled to the first transmitting coil and configured to drive the first transmitting coil,
the first transmitting coil being configured, when the device is in proximity to a person's ear and the first transmitting coil is driven with a time-varying electric current providable by the drive circuit, to generate at least part of a magnetic field providing a time-varying magnetic flux across an auricular branch of a Vagus Nerve (ABVN) sufficient to activate the ABVN.

2. The stimulation device of claim 1, further comprising a second transmitting coil, the first and second transmitting coils being positionable on different sides of a part of the person's ear and configured, when positioned on the different sides of the part of the person's ear and driven with respective time-varying electric currents, to generate at least part of the magnetic field.

3. The stimulation device of claim 2, wherein an axis of the first transmitting coil is aligned or align-able with an axis of the second transmitting coil.

4. The stimulation device of claim 2, wherein an axis of the first transmitting coil and an axis of the second transmitting coil are set or adjustable to intersect and form an angle between about 90 degrees and about 180 degrees.

5. The stimulation device of claim 2, wherein the device is configured to directionally control at least part of the magnetic field by controlling one or both of the relative positions and the relative orientations of the first and second transmitting coils with respect to each other.

6. The stimulation device of claim 2, wherein the device is configured to generate and directionally control at least part of the magnetic field by:
driving the first transmitting coil and selectively driving the second transmitting coil; or
driving the first transmitting coil and differentially driving the second transmitting coil.

7. The stimulation device of claim 1, further comprising a ferrite piece, the ferrite piece being positionable on a different side of a part of the person's ear to the first transmitting coil such that a magnitude of the magnetic field across the ABVN is greater than when the ferrite piece is excluded.

8. The stimulation device of claim 7, wherein the ferrite piece is shaped to at least partially extend around the part of the person's ear.

9. The stimulation device of claim 1, wherein the first transmitting coil is shaped such that two ends of the first transmitting coil are positionable on different sides of a part of the person's ear.

10. The stimulation device of claim 9, wherein the first transmitting coil has an open-ringed or horseshoe shape.

11. The stimulation device of claim 9, wherein the device is configured such that a distance between the two ends of the first transmitting coil is adjustable.

12. The stimulation device of claim 1, further comprising a second transmitting coil and a third transmitting coil, the second and third transmitting coils being positionable on a different side of a part of the person's ear to the first transmitting coil.

13. The stimulation device of claim 12, wherein the device is configured to generate and directionally control at least part of the magnetic field by:
driving the first transmitting coil and selectively driving the second transmitting coil or the third transmitting coil; or
driving the first transmitting coil and differentially driving the second transmitting coil and the third transmitting coil.

14. The stimulation device of claim 13, wherein the device is configured to differentially drive the second and third transmitting coils by at least one selected from:
driving the second and third transmitting coils with respective time-varying electric currents that have different amplitudes; and
driving the second and third transmitting coils with respective time-varying electric currents that are out-of-phase from each other.

15. The stimulation device of claim 12, further comprising a fourth transmitting coil positionable on a same side of the part of the person's ear as the first transmitting coil.

16. The stimulation device of claim 1, wherein the device is configured to fixedly couple to the person's ear.

17. The stimulation device of claim 16, wherein the device comprises a clamp mechanism configured to clamp onto a part of the person's ear.

18. The stimulation device of claim 17, wherein the clamp mechanism comprises first and second parts positionable on different sides of the part of the person's ear that the clamp is configured to clamp onto, and wherein the first transmitting coil is at least partially provided in the first part.

19. The stimulation device of claim 1, further comprising:
a control circuit configured to control operations of the drive circuit,
wherein the drive circuit is electrically coupled to the first transmitting coil and configured to provide the time-varying electric current to the first transmitting coil.

20. The stimulation device of claim 19, wherein the control circuit is configured to cause the drive circuit to provide a current pulse or an alternating current (AC) to the first transmitting coil.

21. The stimulation device of claim 1, wherein the device is configured to generate at least part of the magnetic field by discharging one or more capacitors through the first transmitting coil.

22. The stimulation device of claim 21, wherein the device is configured to generate magnetic pulses having periods of about 10 milliseconds or less.

23. The stimulation device of claim 21, wherein the first transmitting coil and the one or more capacitors form at least part of an inductor-capacitor (LC) circuit.

24. The stimulation device of claim 1, further comprising:
a temperature sensor to measure at least one selected from a temperature of the first transmitting coil and a temperature of a portion of the person's ear proximal to the first transmitting coil; and
a control circuit communicatively coupled to the temperature sensor and configured to control operations of the drive circuit, the control circuit being configured to stop the drive circuit from driving of the first transmitting coil in response to a temperature threshold being exceeded.

25. The stimulation device of claim 24, wherein the temperature threshold is exceeded when the temperature of the portion of the person's ear exceeds about 40 degrees centigrade, or when the temperature of the portion of the person's ear increases by more than about 2 degrees centigrade.

26. The stimulation device of claim 24, wherein the temperature threshold is exceeded when the temperature of the first transmitting coil exceeds about 40 degrees centigrade, or when the temperature of the first transmitting coil increases by more than about 2 degrees centigrade.

27. A stimulation device, the device being configured to couple to a person's ear and comprising a first transmitting coil positioned and oriented such that, when the device is coupled to the person's ear, an axis of the first transmitting coil crosses an auricular branch of a Vagus Nerve (ABVN) of the person.

28. The stimulation device of claim 27, further comprising a second transmitting coil positionable on a different side of a part of the person's ear to the first transmitting coil.

29. The stimulation device of claim 27, wherein the device comprises a clamp mechanism configured to clamp onto a part of the person's ear.

30. The stimulation device of claim 29, wherein the clamp mechanism comprises first and second parts positionable on different sides of the part of the person's ear that the clamp is configured to clamp onto, and wherein the first transmitting coil is at least partially provided in the first part.

31. A method of stimulating an auricular branch of a Vagus Nerve (ABVN), the method comprising generating a magnetic field across the ABVN to activate the ABVN.

32. The method of claim 31, wherein the generating the magnetic field comprises driving a first transmitting coil positioned on a first side of a part of a person's ear with a time-varying electric current.

33. The method of claim 32, wherein the generating the magnetic field further comprises driving a second transmitting coil positioned on a second side of the part of the person's ear different from the first side with a time-varying electric current.

34. The method of claim 32, wherein the generating the magnetic field further comprises directionally controlling the magnetic field by:
selectively driving a second transmitting coil or a third transmitting coil with a time-varying electric current, the second and third transmitting coils being positioned on a second side of the part of the person's ear different from the first side, or
differentially driving a second transmitting coil and a third transmitting coil with respective time-varying electric currents, the second and third transmitting coils being positioned on a second side of the part of the person's ear different from the first side.

35. The method of claim 34, wherein the second and third transmitting coils are differentially driven by at least one selected from:
driving the second and third transmitting coils with respective time-varying electric currents that have different amplitudes; and
driving the second and third transmitting coils with respective time-varying electric currents that are out-of-phase from each other.

36. The method of claim 32, wherein a ferrite piece is positioned on a second side of the part of the person's ear different from the first side during the generating the magnetic field and such that a magnitude of the magnetic field across the ABVN is increased compared to when the ferrite piece is excluded.

37. The method of claim 31, wherein the generating the magnetic field comprises driving a first transmitting coil with a time-varying electric current, the first transmitting coil being shaped and positioned such that a first end of the first transmitting coil is on a first side of a part of a person's ear and a second end of the first transmitting coil is on a second side of a part of the person's ear different from the first side.

38. The method of claim 32, wherein the driving the first transmitting coil comprises providing an electric current pulse to the first transmitting coil or providing an alternating current (AC) to the first transmitting coil.

39. The method of claim 32, further comprising measuring a temperature of a portion of the person's ear proximal to the first transmitting coil and stopping the driving of the first transmitting coil in response to determining that a temperature threshold is exceeded.

40. The method of claim 39, wherein the temperature threshold is exceeded when the temperature of the portion of the person's ear exceeds about 40 degrees centigrade or when the temperature of the portion of the person's ear increases by more than about 2 degrees centigrade.

41. The method of claim 32, further comprising measuring a temperature of the first transmitting coil and stopping the driving of the first transmitting coil when a temperature threshold is exceeded.
